(19) United States
(12) Patent Application Publication
(10) Pub. No.: US 2025/0127383 A1
(43) Pub. Date: Apr. 24, 2025

(54) FLEXIBLE ELONGATE DEVICES HAVING ARTICULABLE BODY PORTION SUPPORT STRUCTURES

(71) Applicant: INTUITIVE SURGICAL OPERATIONS, INC., Sunnyvale, CA (US)

(72) Inventors: David Solum, Santa Cruz, CA (US); Lucas Gordon, Santa Barbara, CA (US); Christopher Major, Santa Clara, CA (US); Joseph Kemp, San Jose, CA (US); Russell Field, Pacifica, CA (US)

(21) Appl. No.: 18/924,326

(22) Filed: Oct. 23, 2024

Related U.S. Application Data
(60) Provisional application No. 63/592,865, filed on Oct. 24, 2023.

(51) Int. Cl. A61B 1/008; A61B 1/005

(52) U.S. Cl. CPC A61B 1/008; A61B 1/0057

(57) ABSTRACT

Flexible elongate devices include an elongate body having an articulable body portion and an axial support structure within the articulable body portion. The axial support structure can include links longitudinally stacked on one another, where each of the links includes a body defining a plurality of pull wire openings, an outwardly protruding hinge, and a socket. The socket is circumferentially offset relative to the hinge and configured to receive the hinge of one of the links therein. The flexible elongate device can include a distal member disposed at a distal end of the axial support structure, a proximal member disposed at a proximal end of the axial support structure, and a braid sheath surrounding the axial support structure within the articulable body portion, where ends of the braid sheath are coupled to the distal and proximal members.

### CROSS-REFERENCE TO RELATED APPLICATION

This application claims the priority benefit of U.S. Provisional Application No. 63/592,865, filed Oct. 24, 2023, which is hereby incorporated by reference herein in its entirety.

### FIELD

Disclosed embodiments relate to flexible elongate devices.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. One such minimally invasive technique is to use a flexible and/or steerable elongate device, such as a flexible catheter, that can be inserted into anatomic passageways and navigated toward a region of interest within the patient anatomy.

### SUMMARY

The following presents a simplified summary of various examples described herein and is not intended to identify key or critical elements or to delineate the scope of the claims.

In accordance with some examples, a flexible elongate device is described herein that includes an elongate body having an articulable body portion and an axial support structure within the articulable body portion. The axial support structure includes a plurality of links longitudinally stacked on one another. Each of the plurality of links includes a body having a first end and a second end, where the body defines a plurality of pull wire openings extending therethrough to receive a plurality of pull wires that control articulation of the articulable body portion. Each of the plurality of links further includes a hinge protruding outwardly from the first end of the body and a socket at the second end of the body, the socket being circumferentially offset relative to the hinge and configured to receive the hinge of one of the links therein. The body can further define pull wire relief recesses in axial ends of the body aligned with ones of the plurality of pull wire openings transverse to a bending axis of an adjacent hinge and socket interface.

In accordance with some examples, a flexible elongate device is described herein that includes an elongate body having an articulable body portion, an axial support structure within the articulable body portion, a distal member disposed at a distal end of the axial support structure, a proximal member disposed at a proximal end of the axial support structure, and a braid sheath surrounding the axial support structure within the articulable body portion, where ends of the braid sheath are coupled to the distal and proximal members.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a medical system according to some embodiments.

FIG. 2A is a simplified diagram of a medical instrument system according to some embodiments.

FIG. 2B is a simplified diagram of a medical instrument including a medical tool within an elongate device according to some embodiments.

FIGS. 3A and 3B are simplified diagrams of side views of a patient coordinate space including a medical instrument mounted on an insertion assembly according to some embodiments.

FIG. 4A is a sectional perspective view of a flexible elongate device according to some embodiments.

FIG. 4B is a sectional perspective view of the flexible elongate device of FIG. 4A showing a braid sheath for an axial support structure according to some embodiments.

FIG. 4C is a sectional perspective view of the flexible elongate device of FIG. 4B showing the braid sheath welded to a distal member according to some embodiments.

FIG. 4D is a sectional perspective view of the flexible elongate device of FIG. 4B showing the braid sheath welded to a proximal member according to some embodiments.

FIG. 5A is a sectional perspective view of a flexible elongate device showing an axial support structure according to some embodiments.

FIG. 5B is a top perspective view of a link for the axial support structure of the flexible elongate device of FIG. 5A according to some embodiments.

FIG. 5C is a bottom perspective view of a link for the axial support structure of the flexible elongate device of FIG. 5A according to some embodiments.

FIG. 5D is a perspective view of stacked links for the axial support structure of the flexible elongate device of FIG. 5A according to some embodiments.

FIG. 5E is a cross-sectional view of stacked links for the axial support structure of the flexible elongate device of FIG. 5A showing first example pull wire relief features according to some embodiments.

FIG. 5F is a cross-sectional view of stacked links for the axial support structure of the flexible elongate device of FIG. 5A showing second example pull wire relief features according to some embodiments.

FIG. 5G is a cross-sectional view of stacked links for the axial support structure of the flexible elongate device of FIG. 5A showing component line opening configurations according to some embodiments.

FIG. 5H is a sectional perspective view of a distal member of the flexible elongate device of FIG. 5A according to some embodiments.

FIG. 6 is an exploded perspective view of a second example distal member for a flexible elongate device according to some embodiments.

FIG. 7 is an exploded perspective view of a third example distal member for a flexible elongate device according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

Flexible elongate devices that include articulable body portions may include an axial support structure that functions to preserve a neutral axis length for the articulable body portion during articulation thereof, as well as support the articulable body portion against axial loads generated during manipulation of the articulable body portion. For example, the axial support structure may prevent or reduce distortion, compression, and/or collapse of the articulable body portion under axial loads or other types of forces (e.g., external force).

Some axial support structures may be formed by cutting away material from a tubular structure, which can have a correspondingly high cost of materials and manufacture. The axial support structures provided herein include links that may have a lower material and manufacturing cost than prior axial support structures.

The axial support structures as provided herein include a plurality of links that are stacked along a longitudinal axis of the flexible elongate device. Each link includes a hinge on one end and a socket on an opposite end, such that the hinge of one link is received within the socket of an adjacent link. A bending axis of the hinge extends laterally across the link, which allows the axial support structure to bend along the bending axis lateral to the neutral axis. In some examples, the hinge and socket of each link can be circumferentially offset relative to one another (e.g., a 60 degree offset, a 72 degree offset, a 90 degree offset, a 120 degree offset, etc.) to allow the axial support structure and therefore the articulable body portion to bend along multiple axes (e.g., pitch and yaw). In some examples, the hinge can include inwardly tapering surfaces, helping align the stacked links to a common centerline.

In some examples, articulation of the articulable body portion of the flexible elongate device can be achieved by manipulation of pull wires coupled to a control structure (e.g., a control ring or tip portion) disposed within the articulable body portion. In these examples, the links can include pull wire openings extending therethrough, such that the pull wires can pass through the plurality of links. In some examples, links can be configured to reduce stress and/or friction on the pull wires caused by articulation of the links relative to one another. For example, the links can define a recess in an end surface aligned with the pull wire opening. Adjacent links can have aligned recesses to provide an increased bend radius for the pull wire to extend between the links during articulation. Recesses can be helpful for pull wires extending through the links spaced from a bending axis for a particular articulation. In another example, the pull wire openings can have an expanding or increased inner dimension (e.g., transitioning from a circular cross-section to an elongate, slot cross-section) as the pull wire opening extends to the end surface of the link. Adjacent links can have aligned pull wire openings having expanding or increased inner dimensions to provide an increased bend radius for the pull wire to extend between the links during an articulation. Pull wire openings having expanding or increased inner dimensions can be helpful for pull wires extending through the links adjacent to a bending axis for a particular articulation.

In some examples, the flexible elongate device may include an inner body member, which can define a lumen for the flexible elongate device. In these examples, the links may be annular having an inner diameter sized to receive the inner body member therethrough. Furthermore, the flexible elongate device may include one or more component lines (e.g., wires, sensors, conductors, etc.) that extend through the articulable body portion of the flexible elongate device. As such, each link of the axial support structure can include one or more component slots to receive the component lines therethrough. In one example, each link can include a plurality of component slots having a spaced configured around the circumference of the link, such that when the link is rotated relative to an adjacent link due to the hinge/socket offset, one of the component slots aligns with one of the component slots of the adjacent link to provide a continuous longitudinal path for the component line through the axial support structure. In a further example, the flexible elongate device can include two component lines and three component slots having a spaced configuration around the circumference of the link, such that when the link is rotated relative to an adjacent link due to the hinge/socket offset, two of the component slots align with two of the component slots of the adjacent link to provide continuous longitudinal paths for the component lines through the axial support structure.

In some examples, the articulable body portion of the flexible elongate device can include a braid sheath disposed over an axial support structure of the articulable body portion. The braid sheath can support the articulable body portion, and the axial support structure thereof, against tensile and, in some embodiments, compressive forces, which can result from handling, lubrication, sterilization, reprocessing, etc. The articulable body portion can include a proximal member (e.g., a stopper) and a distal member (e.g. a control structure), with the axial support structure therebetween. Ends of the braid sheath are laser welded to the proximal and distal members so that the braid sheath extends over the articulable body portion and resists tensile forces. Welds securing the braid sheath to the proximal/distal member can be located at cross-over points of the braid material.

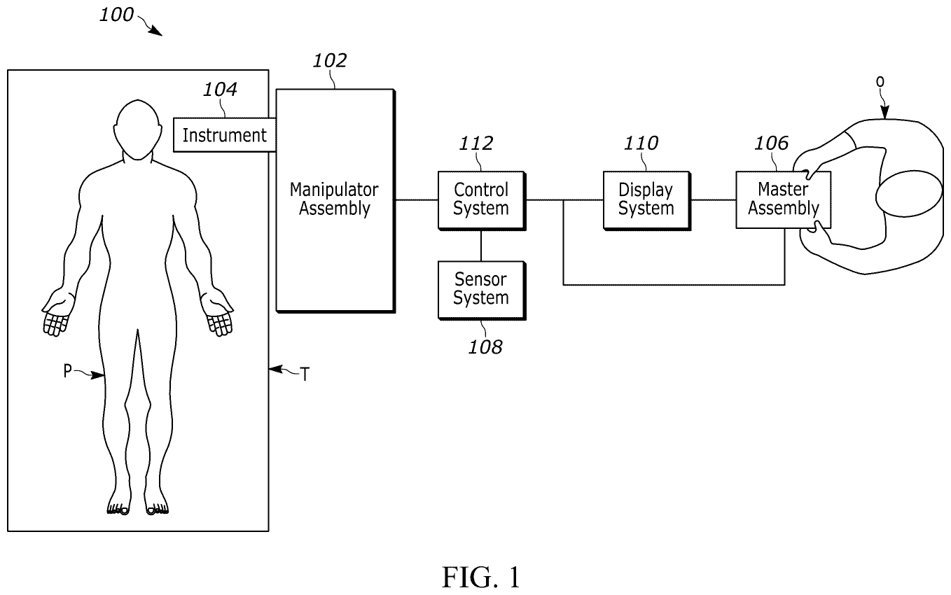

FIG. 1 is a simplified diagram of a medical system 100 according to some embodiments. The medical system 100 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 1, medical system 100 may include a manipulator assembly 102 that controls the operation of a medical instrument 104 in performing various procedures on a patient P. Medical instrument 104 may extend into an internal site within the body of patient P via an opening in the body of patient P. The manipulator assembly 102 may be teleoperated, non-teleoperated, or a hybrid teleoperated and non-teleoperated assembly with one or more degrees of freedom of motion that may be motorized and/or one or more degrees of freedom of motion that may be non-motorized (e.g., manually operated). The manipulator assembly 102 may be mounted to and/or positioned near a patient table T. A master assembly 106 allows an operator O (e.g., a surgeon, a clinician, a physician, or other user) to control the manipulator assembly 102. In some examples, the master assembly 106 allows the operator O to view the procedural site or other graphical or informational displays. In some examples, the manipulator assembly 102 may be excluded from the medical system 100 and the instrument 104 may be controlled directly by the operator O. In some examples, the manipulator assembly 102 may be manually controlled by the operator O. Direct operator control may include various handles and operator interfaces for hand-held operation of the instrument 104.

The master assembly 106 may be located at a surgeon's console which is in proximity to (e.g., in the same room as) a patient table T on which patient P is located, such as at the side of the patient table T. In some examples, the master assembly 106 is remote from the patient table T, such as in in a different room or a different building from the patient table T. The master assembly 106 may include one or more control devices for controlling the manipulator assembly 102. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 102 supports the medical instrument 104 and may include a kinematic structure of links that provide a set-up structure. The links may include one or more non-servo controlled links (e.g., one or more links that may be manually positioned and locked in place) and/or one or more servo controlled links (e.g., one or more links that may be controlled in response to commands, such as from a control system 112). The manipulator assembly 102 may include a plurality of actuators (e.g., motors) that drive inputs on the medical instrument 104 in response to commands, such as from the control system 112. The actuators may include drive systems that move the medical instrument 104 in various ways when coupled to the medical instrument 104. For example, one or more actuators may advance medical instrument 104 into a naturally or surgically created anatomic orifice. Actuators may control articulation of the medical instrument 104, such as by moving the distal end (or any other portion) of medical instrument 104 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 104, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 104.

The medical system 100 may include a sensor system 108 with one or more sub-systems for receiving information about the manipulator assembly 102 and/or the medical instrument 104. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 104; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 104 or from some other location; and/or actuator position sensors such as resolvers, encoders, potentiometers, and the like that describe the rotation and/or orientation of the actuators controlling the medical instrument 104.

The medical system 100 may include a display system 110 for displaying an image or representation of the procedural site and the medical instrument 104. Display system 110 and master assembly 106 may be oriented so physician O can control medical instrument 104 and master assembly 106 with the perception of telepresence.

In some embodiments, the medical instrument 104 may include a visualization system, which may include an image capture assembly that records a concurrent or real-time image of a procedural site and provides the image to the operator O through one or more displays of display system 110. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 104. Additionally or alternatively, a separate endoscope, attached to a separate manipulator assembly, may be used with medical instrument 104 to image the procedural site. The visualization system may be implemented as hardware, firmware, software or a combination thereof which interact with or are otherwise executed by one or more computer processors, such as of the control system 112.

Display system 110 may also display an image of the procedural site and medical instruments, which may be captured by the visualization system. In some examples, the medical system 100 provides a perception of telepresence to the operator O. For example, images captured by an imaging device at a distal portion of the medical instrument 104 may be presented by the display system 110 to provide the perception of being at the distal portion of the medical instrument 104 to the operator O. The input to the master assembly 106 provided by the operator O may move the distal portion of the medical instrument 104 in a manner that corresponds with the nature of the input (e.g., distal tip turns right when a trackball is rolled to the right) and results in corresponding change to the perspective of the images captured by the imaging device at the distal portion of the medical instrument 104. As such, the perception of telepresence for the operator O is maintained as the medical instrument 104 is moved using the master assembly 106. The operator O can manipulate the medical instrument 104 and hand controls of the master assembly 106 as if viewing the workspace in substantially true presence, simulating the experience of an operator that is physically manipulating the medical instrument 104 from within the patient anatomy.

In some examples, the display system 110 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 110 may display a virtual image that is generated based on tracking the location of medical instrument 104. For example, the tracked location of the medical instrument 104 may be registered (e.g., dynamically referenced) with the model generated using the pre-operative or intra-operative images, with different portions of the model correspond with different locations of the patient anatomy. As the medical instrument 104 moves through the patient anatomy, the registration is used to determine portions of the model corresponding with the location and/or perspective of the medical instrument 104 and virtual images are generated using the determined portions of the model. This may be done to present the operator O with virtual images of the internal procedural site from viewpoints of medical instrument 104 that correspond with the tracked locations of the medical instrument 104.

The medical system 100 may also include the control system 112, which may include processing circuitry that implements the some or all of the methods or functionality discussed herein. The control system 112 may include at least one memory and at least one processor for controlling the operations of the manipulator assembly 102, the medical instrument 104, the master assembly 106, the sensor system 108, and/or the display system 110. Control system 112 may include instructions (e.g., a non-transitory machine-readable medium storing the instructions) that when executed by the at least one processor, configures the one or more processors to implement some or all of the methods or functionality discussed herein. While the control system 112 is shown as a single block in FIG. 1, the control system 112 may include two or more separate data processing circuits with one portion of the processing being performed at the manipulator assembly 102, another portion of the processing being performed at the master assembly 106, and/or the like. In some examples, the control system 112 may include other types of processing circuitry, such as application-specific integrated circuits (ASICs) and/or field-programmable gate array (FPGAs). The control system 112 may be implemented using hardware, firmware, software, or a combination thereof.

In some examples, the control system 112 may receive feedback from the medical instrument 104, such as force and/or torque feedback. Responsive to the feedback, the control system 112 may transmit signals to the master assembly 106. In some examples, the control system 112 may transmit signals instructing one or more actuators of the manipulator assembly 102 to move the medical instrument 104. In some examples, the control system 112 may transmit informational displays regarding the feedback to the display system 110 for presentation or perform other types of actions based on the feedback.

The control system 112 may include a virtual visualization system to provide navigation assistance to operator O when controlling the medical instrument 104 during an image-guided medical procedure. Virtual navigation using the virtual visualization system may be based upon an acquired pre-operative or intra-operative dataset of anatomic passageways of the patient P. The control system 112 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 108 that is used to compute an (e.g., approximate) location of the medical instrument 104 with respect to the anatomy of patient P. The sensor system 108 may be used to register and display the medical instrument 104 together with the pre-operatively or intra-operatively recorded images. For example, PCT Publication WO 2016/191298 (published Dec. 1, 2016 and titled “Systems and Methods of Registration for Image Guided Surgery”), which is incorporated by reference herein in its entirety, discloses example systems.

During a virtual navigation procedure, the sensor system 108 may be used to compute the (e.g., approximate) location of the medical instrument 104 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Patent No. 8,900,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 100 may further include operations and support systems (not shown) such as illumination systems, steering control systems, irrigation systems, and/or suction systems. In some embodiments, the medical system 100 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

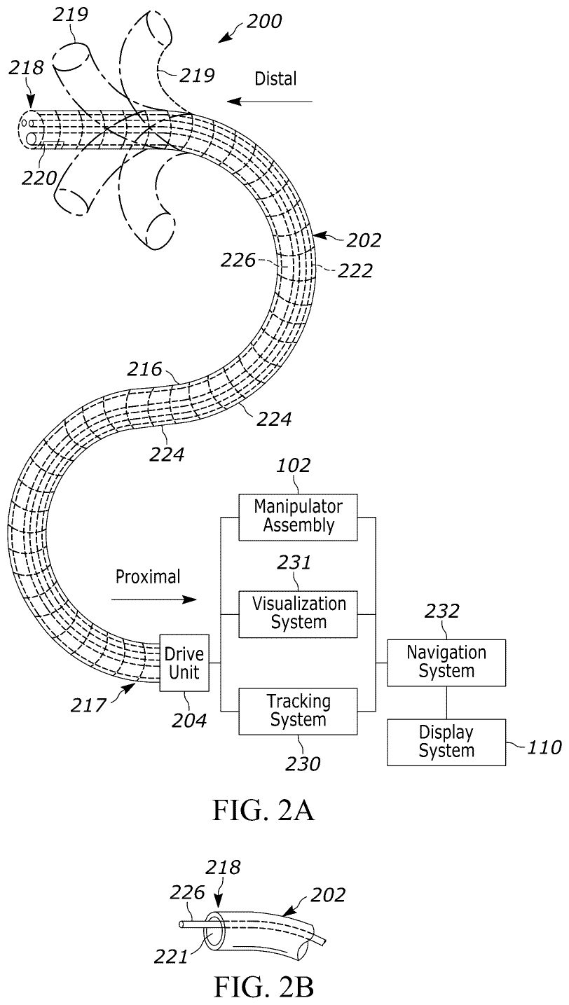

FIG. 2A is a simplified diagram of a medical instrument system 200 according to some embodiments. The medical instrument system 200 includes a flexible elongate device 202 (also referred to as elongate device 202), a drive unit 204, and a medical tool 226 that collectively is an example of a medical instrument 104 of a medical system 100. The medical system 100 may be a teleoperated system, a non-teleoperated system, or a hybrid teleoperated and non-teleoperated system, as explained with reference to FIG. 1. A visualization system 231, tracking system 230, and navigation system 232 are also shown in FIG. 2A and are example components of the control system 112 of the medical system 100. In some examples, the medical instrument system 200 may be used for non-teleoperational exploratory procedures or in procedures involving traditional manually operated medical instruments, such as endoscopy. The medical instrument system 200 may be used to gather (e.g., measure) a set of data points corresponding to locations within anatomic passageways of a patient, such as patient P.

The elongate device 202 is coupled to the drive unit 204. The elongate device 202 includes a channel 221 through which the medical tool 226 may be inserted. The elongate device 202 navigates within patient anatomy to deliver the medical tool 226 to a procedural site. The elongate device 202 includes a flexible body 216 having a proximal end 217 and a distal end 218. In some examples, the flexible body 216 may have an approximately 3 mm outer diameter. Other flexible body outer diameters may be larger or smaller.

Medical instrument system 200 may include the tracking system 230 for determining the position, orientation, speed, velocity, pose, and/or shape of the flexible body 216 at the distal end 218 and/or of one or more segments 224 along flexible body 216, as will be described in further detail below. The tracking system 230 may include one or more sensors and/or imaging devices. The flexible body 216, such as the length between the distal end 218 and the proximal end 217, may include multiple segments 224. The tracking system 230 may be implemented using hardware, firmware, software, or a combination thereof. In some examples, the tracking system 230 is part of control system 112 shown in FIG. 1.

Tracking system 230 may track the distal end 218 and/or one or more of the segments 224 of the flexible body 216 using a shape sensor 222. The shape sensor 222 may include an optical fiber aligned with the flexible body 216 (e.g., provided within an interior channel of the flexibly body 216 or mounted externally along the flexible body 216). In some examples, the optical fiber may have a diameter of approximately 200 μm. In other examples, the diameter may be larger or smaller. The optical fiber of the shape sensor 222 may form a fiber optic bend sensor for determining the shape of flexible body 216. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sep. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 216 may be determined using other techniques. For example, a history of the position and/or pose of the distal end 218 of the flexible body 216 can be used to reconstruct the shape of flexible body 216 over an interval of time (e.g., as the flexible body 216 is advanced or retracted within a patient anatomy). In some examples, the tracking system 230 may alternatively and/or additionally track the distal end 218 of the flexible body 216 using a position sensor system 220. Position sensor system 220 may be a component of an EM sensor system with the position sensor system 220 including one or more position sensors. Although the position sensor system 220 is shown as being near the distal end 218 of the flexible body 216 to track the distal end 218, the number and location of the position sensors of the position sensor system 220 may vary to track different regions along the flexible body 216. In one example, the position sensors include conductive coils that may be subjected to an externally generated electromagnetic field. Each coil of position sensor system 220 may produce an induced electrical signal having characteristics that depend on the position and orientation of the coil relative to the externally generated electromagnetic field. The position sensor system 220 may measure one or more position coordinates and/or one or more orientation angles associated with one or more portions of flexible body 216. In some examples, the position sensor system 220 may be configured and positioned to measure six degrees of freedom, e.g., three position coordinates X, Y, Z and three orientation angles indicating pitch, yaw, and roll of a base point. In some examples, the position sensor system 220 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999 and titled “Six-Degree of Freedom Tracking System Having a Passive Transponder on the Object Being Tracked”), which is incorporated by reference herein in its entirety.

In some embodiments, the tracking system 230 may alternately and/or additionally rely on a collection of pose, position, and/or orientation data stored for a point of an elongate device 202 and/or medical tool 226 captured during one or more cycles of alternating motion, such as breathing. This stored data may be used to develop shape information about the flexible body 216. In some examples, a series of position sensors (not shown), such as EM sensors like the sensors in position sensor 220 or some other type of position sensors may be positioned along the flexible body 216 and used for shape sensing. In some examples, a history of data from one or more of these position sensors taken during a procedure may be used to represent the shape of elongate device 202, particularly if an anatomic passageway is generally static.

FIG. 2B is a simplified diagram of the medical tool 226 within the elongate device 202 according to some embodiments. The flexible body 216 of the elongate device 202 may include the channel 221 sized and shaped to receive the medical tool 226. In some embodiments, the medical tool 226 may be used for procedures such as diagnostics, imaging, surgery, biopsy, ablation, illumination, irrigation, suction, electroporation, etc. Medical tool 226 can be deployed through channel 221 of flexible body 216 and operated at a procedural site within the anatomy. Medical instrument 226 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 226 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 226 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 221 when the biopsy tool is within the channel 221. The medical tool 226 may be an image capture probe that includes a distal portion with a stereoscopic or monoscopic camera that may be placed at or near the distal end 218 of flexible body 216 for capturing images (e.g., still or video images). The captured images may be processed by the visualization system 231 for display and/or provided to the tracking system 230 to support tracking of the distal end 218 of the flexible body 216 and/or one or more of the segments 224 of the flexible body 216. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 231. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 216 of the elongate device 202 to facilitate visual navigation of the elongate device 202 to a procedural site and then is replaced within the flexible body 216 with another type of medical tool 226 that performs the procedure. In some examples, the image capture probe may be within the flexible body 216 of the elongate device 202 along with another type of medical tool 226 to facilitate simultaneous image capture and tissue intervention, such as within the same channel 221 or in separate channels. A medical tool 226 may be advanced from the opening of the channel 221 to perform the procedure (or some other functionality) and then retracted back into the channel 221 when the procedure is complete. The medical tool 226 may be removed from the proximal end 217 of the flexible body 216 or from another optional instrument port (not shown) along flexible body 216.

In some examples, the elongate device 202 may include integrated imaging capability rather than utilize a removable image capture probe. For example, the imaging device (or fiber-optic bundle) and the light emitters may be located at the distal end 218 of the elongate device 202. The flexible body 215 may include one or more dedicated channels that carry the cable(s) and/or optical fiber(s) between the distal end 218 and the visualization system 231. Here, the medical instrument system 200 can perform simultaneous imaging and tool operations.

In some examples, the medical tool 226 is capable of controllable articulation. The medical tool 226 may house cables (which may also be referred to as pull wires), linkages, or other actuation controls (not shown) that extend between its proximal and distal ends to controllably bend the distal end of medical tool 226, such as discussed herein for the flexible elongate device 202. The medical tool 226 may be coupled to a drive unit 204 and the manipulator assembly 102. In these examples, the elongate device 202 may be excluded from the medical instrument system 200 or may be a flexible device that does not have controllable articulation. Steerable instruments or tools, applicable in some embodiments, are further described in detail in U.S. Pat. No. 7,316,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sep. 30, 2008 and titled “Passive Preload and Capstan Drive for Surgical Instruments”), which are incorporated by reference herein in their entireties.

The flexible body 216 of the elongate device 202 may also or alternatively house cables, linkages, or other steering controls (not shown) that extend between the drive unit 204 and the distal end 218 to controllably bend the distal end 218 as shown, for example, by broken dashed line depictions 219 of the distal end 218 in FIG. 2A. In some examples, at least four cables are used to provide independent up-down steering to control a pitch of the distal end 218 and left-right steering to control a yaw of the distal end 281. In these examples, the flexible elongate device 202 may be a steerable catheter. Examples of steerable catheters, applicable in some embodiments, are described in detail in PCT Publication WO 2019/018736 (published Jan. 24, 2019 and titled “Flexible Elongate Device Systems and Methods”), which is incorporated by reference herein in its entirety.

In embodiments where the elongate device 202 and/or medical tool 226 are actuated by a teleoperational assembly (e.g., the manipulator assembly 102), the drive unit 204 may include drive inputs that removably couple to and receive power from drive elements, such as actuators, of the teleoperational assembly. In some examples, the elongate device 202 and/or medical tool 226 may include gripping features, manual actuators, or other components for manually controlling the motion of the elongate device 202 and/or medical tool 226. The elongate device 202 may be steerable or, alternatively, the elongate device 202 may be non-steerable with no integrated mechanism for operator control of the bending of distal end 218. In some examples, one or more channels 221 (which may also be referred to as lumens), through which medical tools 226 can be deployed and used at a target anatomical location, may be defined by the interior walls of the flexible body 216 of the elongate device 202.

In some examples, the medical instrument system 200 (e.g., the elongate device 202 or medical tool 226) may include a flexible bronchial instrument, such as a bronchoscope or bronchial catheter, for use in examination, diagnosis, biopsy, and/or treatment of a lung. The medical instrument system 200 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 230 may be sent to the navigation system 232, where the information may be combined with information from the visualization system 231 and/or pre-operatively obtained models to provide the physician, clinician, surgeon, or other operator with real-time position information. In some examples, the real-time position information may be displayed on the display system 110 for use in the control of the medical instrument system 200. In some examples, the navigation system 232 may utilize the position information as feedback for positioning medical instrument system 200. Various systems for using fiber optic sensors to register and display a surgical instrument with surgical images, applicable in some embodiments, are provided in U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety.

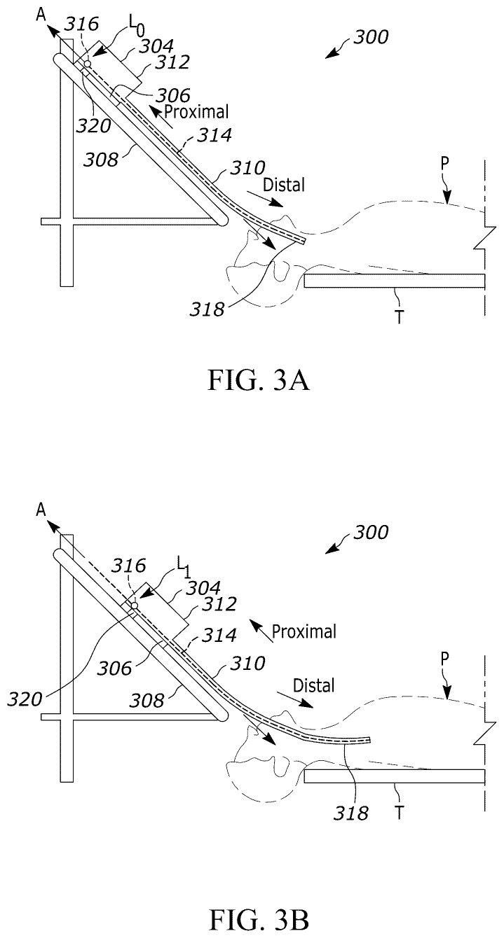

FIGS. 3A and 3B are simplified diagrams of side views of a patient coordinate space including a medical instrument mounted on an insertion assembly according to some embodiments. As shown in FIGS. 3A and 3B, a surgical environment 300 may include a patient P positioned on the patient table T. Patient P may be stationary within the surgical environment 300 in the sense that gross patient movement is limited by sedation, restraint, and/or other means. Cyclic anatomic motion, including respiration and cardiac motion, of patient P may continue. Within surgical environment 300, a medical instrument 304 is used to perform a medical procedure which may include, for example, surgery, biopsy, ablation, illumination, irrigation, suction, or electroporation. The medical instrument 304 may also be used to perform other types of procedures, such as a registration procedure to associate the position, orientation, and/or pose data captured by the sensor system 108 to a desired (e.g., anatomical or system) reference frame. The medical instrument 304 may be, for example, the medical instrument 104. In some examples, the medical instrument 304 may include an elongate device 310 (e.g., a catheter) coupled to an instrument body 312. Elongate device 310 includes one or more channels sized and shaped to receive a medical tool.

Elongate device 310 may also include one or more sensors (e.g., components of the sensor system 108). In some examples, a shape sensor 314 may be fixed at a proximal point 316 on the instrument body 312. The proximal point 316 of the shape sensor 314 may be movable with the instrument body 312, and the location of the proximal point 316 with respect to a desired reference frame may be known (e.g., via a tracking sensor or other tracking device). The shape sensor 314 may measure a shape from the proximal point 316 to another point, such as a distal end 318 of the elongate device 310. The shape sensor 314 may be aligned with the elongate device 310 (e.g., provided within an interior channel or mounted externally). In some examples, the shape sensor 314 may optical fibers used to generate shape information for the elongate device 310.

In some examples, position sensors (e.g., EM sensors) may be incorporated into the medical instrument 304. A series of position sensors may be positioned along the flexible elongate device 310 and used for shape sensing. Position sensors may be used alternatively to the shape sensor 314 or with the shape sensor 314, such as to improve the accuracy of shape sensing or to verify shape information.

Elongate device 310 may house cables, linkages, or other steering controls that extend between the instrument body 312 and the distal end 318 to controllably bend the distal end 318. In some examples, at least four cables are used to provide independent up-down steering to control a pitch of distal end 318 and left-right steering to control a yaw of distal end 318. The instrument body 312 may include drive inputs that removably couple to and receive power from drive elements, such as actuators, of a manipulator assembly.

The instrument body 312 may be coupled to an instrument carriage 306. The instrument carriage 306 may be mounted to an insertion stage 308 that is fixed within the surgical environment 300. Alternatively, the insertion stage 308 may be movable but have a known location (e.g., via a tracking sensor or other tracking device) within surgical environment 300. Instrument carriage 306 may be a component of a manipulator assembly (e.g., manipulator assembly 102) that couples to the medical instrument 304 to control insertion motion (e.g., motion along an insertion axis A) and/or motion of the distal end 318 of the elongate device 310 in multiple directions, such as yaw, pitch, and/or roll. The instrument carriage 306 or insertion stage 308 may include actuators, such as servomotors, that control motion of instrument carriage 306 along the insertion stage 308.

A sensor device 320, which may be a component of the sensor system 108, may provide information about the position of the instrument body 312 as it moves relative to the insertion stage 308 along the insertion axis A. The sensor device 320 may include one or more resolvers, encoders, potentiometers, and/or other sensors that measure the rotation and/or orientation of the actuators controlling the motion of the instrument carriage 306, thus indicating the motion of the instrument body 312. In some embodiments, the insertion stage 308 has a linear track as shown in FIGS. 3A and 3B. In some embodiments, the insertion stage 308 may have curved track or have a combination of curved and linear track sections.

FIG. 3A shows the instrument body 312 and the instrument carriage 306 in a retracted position along the insertion stage 308. In this retracted position, the proximal point 316 is at a position LO on the insertion axis A. The location of the proximal point 316 may be set to a zero value and/or other reference value to provide a base reference (e.g., corresponding to the origin of a desired reference frame) to describe the position of the instrument carriage 306 along the insertion stage 308. In the retracted position, the distal end 318 of the elongate device 310 may be positioned just inside an entry orifice of patient P. Also in the retracted position, the data captured by the sensor device 320 may be set to a zero value and/or other reference value (e.g., I=0). In FIG. 3B, the instrument body 312 and the instrument carriage 306 have advanced along the linear track of insertion stage 308, and the distal end 318 of the elongate device 310 has advanced into patient P. In this advanced position, the proximal point 316 is at a position L1 on the insertion axis A. In some examples, the rotation and/or orientation of the actuators measured by the sensor device 320 indicating movement of the instrument carriage 306 along the insertion stage 308 and/or one or more position sensors associated with instrument carriage 306 and/or the insertion stage 308 may be used to determine the position L1 of the proximal point 316 relative to the position L0. In some examples, the position L1 may further be used as an indicator of the distance or insertion depth to which the distal end 318 of the elongate device 310 is inserted into the passageway(s) of the anatomy of patient P.

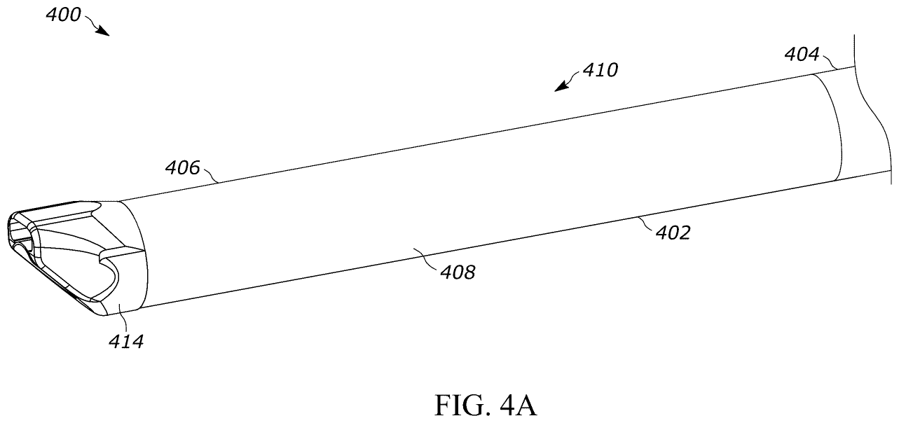

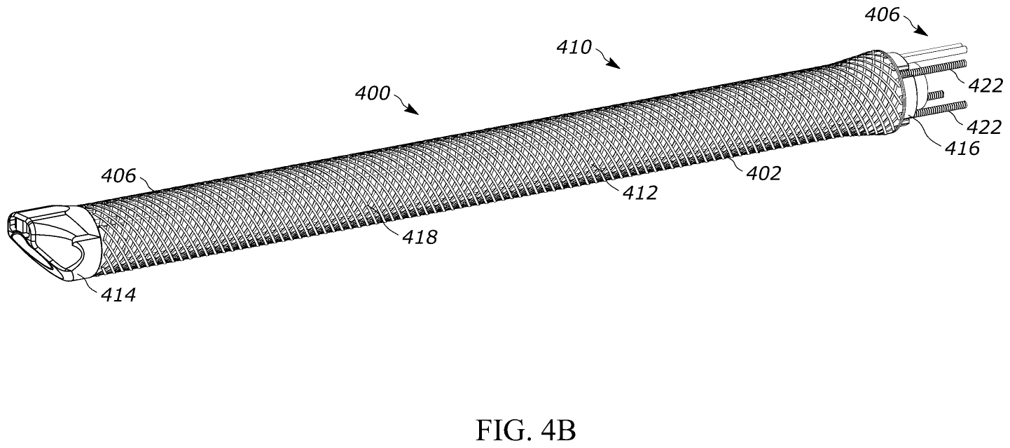

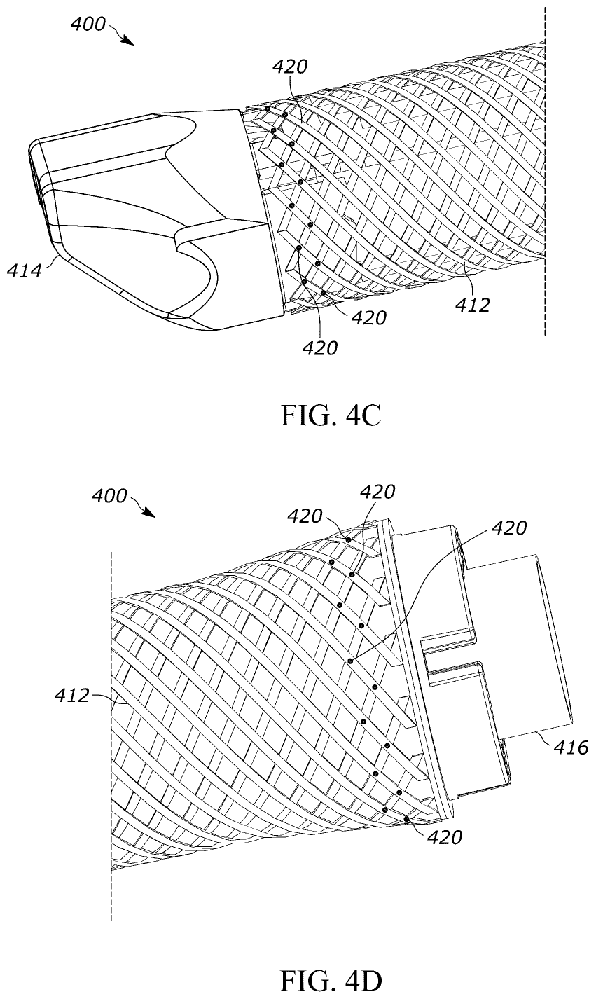

FIGS. 4A-4C illustrate components of a flexible elongate device 400 according to some embodiments. According to some embodiments consistent with FIGS. 1-3B, the flexible elongate device 400 may correspond to the elongate device 202 of the medical instrument system 200.

The flexible elongate device 400 includes a flexible body 402 extending from a proximal section 404 to a distal section 406. It will be understood that the flexible body 402, including the proximal and distal sections 404, 406, can have any desired length. As set forth above, the term “distal” refers to a position that is closer to a procedural site and the term “proximal” refers to a position that is further from the procedural site. As shown in FIG. 4A, the flexible body 420 can include an outer jacket 408 that extends along some or all of a length of the flexible elongate device 400 to provide an outer surface for the flexible elongate device 400. The outer jacket 408 can be made from any suitable polymer, metal, or composite material, including polyurethane of various durometers, e.g., high durometer polyurethanes, or a stiffer material having a hardness or durometer higher than polyurethane. Suitable examples can include nylon, polyetheretherketone (PEEK), and so forth. The outer jacket 408 can be applied by any suitable method, including lamination, extrusion, etc.

The flexible elongate device 400 includes an articulable body portion 410, which in some examples can be located in or form the distal section 406 of the flexible body 402, and an axial support structure 412 within the articulable body portion 410. The axial support structure 412 is configured to bend in response to articulation of the articulable body portion 410 and support the articulable body portion 410 against axial loads generated by articulation. In particular, the axial support structure 412 may prevent or reduce distortion, compression, and/or collapse of the articulable body portion 410 under axial loads.

The flexible elongate device 400 can further include a distal member 414 disposed at a distal end of the axial support structure 412 and a proximal member 416 disposed at a proximal end of the axial support structure 412. In some examples, the flexible elongate device 400 can further include a braid sheath 418 that surrounds the axial support structure 412 within the articulable body portion 410. Ends of the braid sheath 418 are coupled to the distal member 414 and the proximal member 416. The braid sheath 418 can advantageously allow the articulable body portion 410 to withstand tensile forces, such as those generated from preparation (e.g., packaging removal, application of coatings, cleaning, etc.) of the flexible elongate device 400 for use. Further, due to the braid sheath's flexible structure, the braid sheath 418 does not add substantially to a bending stiffness of the articulable body portion 410.

As shown in FIGS. 4B and 4C, the ends of the braid sheath 418 can be coupled to the distal member 414 and the proximal member 416 by a plurality of welds 420. To produce a strong bond, the welds 420 can be disposed at locations that provide intimate contact between the wires or fibers of the braid sheath 418 and the distal/proximal member 414, 416. In the illustrated form, the braid sheath 418 includes wires/fibers braided in an over and under pattern. Due to this, at many points, individual wires/fibers are not in contact with the distal/proximal member 414, 416. To ensure contact, the welds 420 can be disposed over or incorporate the cross-over points of the wires/fibers that are aligned over the distal/proximal member 414/416. Additionally, the welds 420 can be numerous enough and/or disposed such that each wire/fiber of the braid sheath 418 has a weld 420 to both the distal member 414 and the proximal member 416, whether individually welded to the member 414, 416 or as part of the cross-over points. The welds 420 can be disposed in the middle of the cross-over points, in a circle around the cross-over points, at the four corners of the cross-over points, and so forth. The welds 420 can be disposed in a repeating W-shaped pattern, in a diamond pattern, and/or in a zig-zag pattern.

In some examples, the welds 420 can be laser welds. In other examples, the welds 420 can be resistance welds. To create resistance welds, electrodes apply pressure and pass current to melt the wires/fibers that are aligned over the distal/proximal member 414/416.

In some examples, the braid sheath 418 can be formed from stainless steel wires (e.g., 304 stainless steel). In some examples, the braid sheath 418 can be heat treated metal (e.g., heat treated stainless steel) for stress relief to set a shape (e.g., annular) for the braid sheath 418 suitable for welding to the cylindrical outer surfaces of the distal member 414 and the proximal member 416. For example, the braid sheath 418 can be rapidly heated (e.g., up to 1,000 degrees F./538 degrees C. within 10 seconds), such as by induction coil.

As shown in FIGS. 4A-4C, the distal member 414 can include a control structure for the articulable body portion 410 attachable to one or more pull wires, discussed in more detail below, to control articulation of the articulable body portion 410. In some examples, the distal member 414 is a (e.g., most distal) link of an axial support structure. In other examples, the distal member 414 is a one-piece structure that includes the distal tip of the flexible elongate device and the control structure that attaches to pull wires. Further, the proximal member 416 can be a stoppers and/or coupling structure for coil pipes 422, each coil pipe 422 being configured receive a respective pull wire.

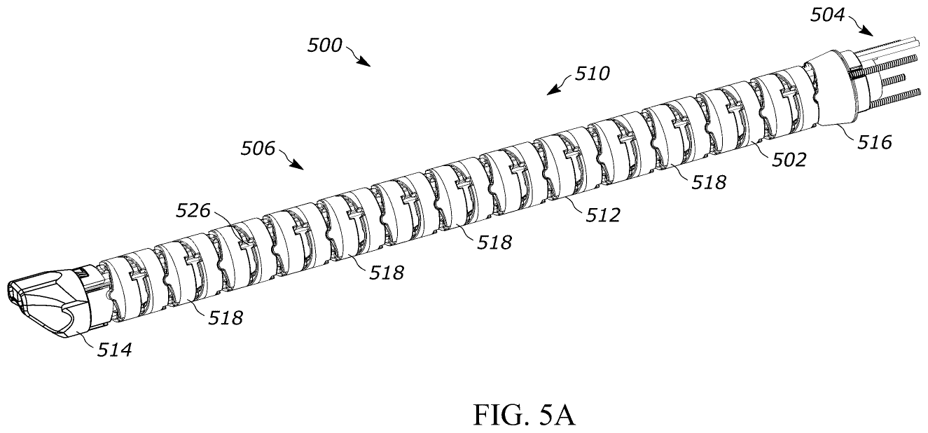

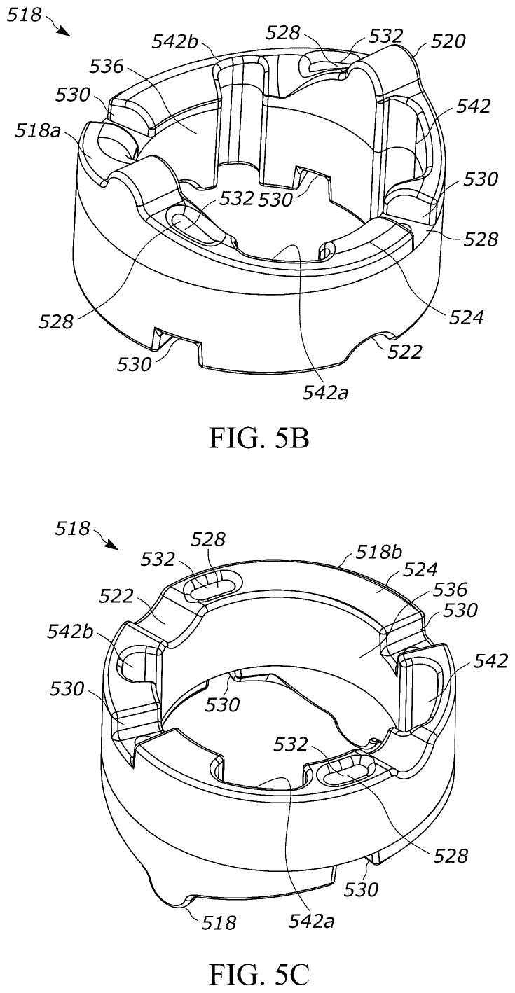

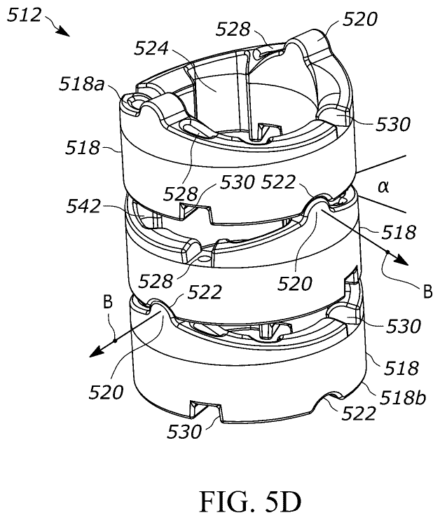

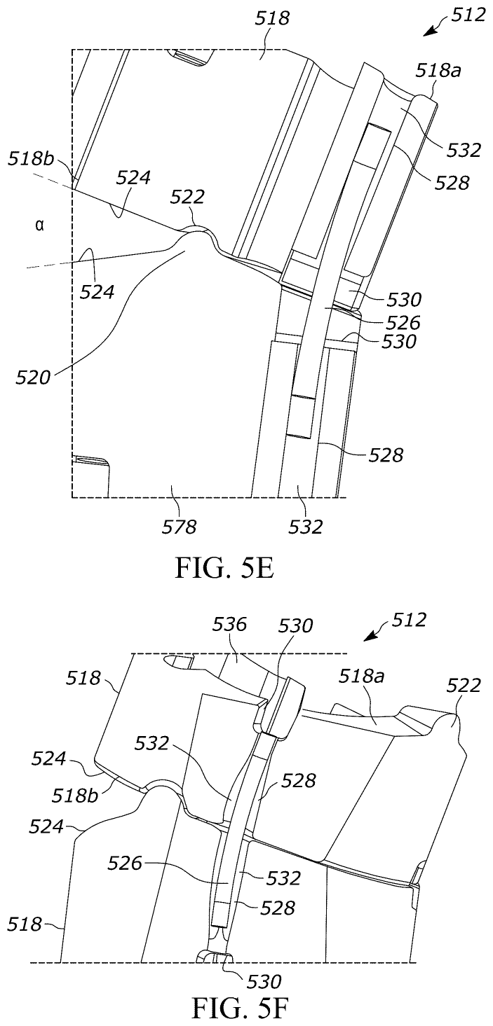

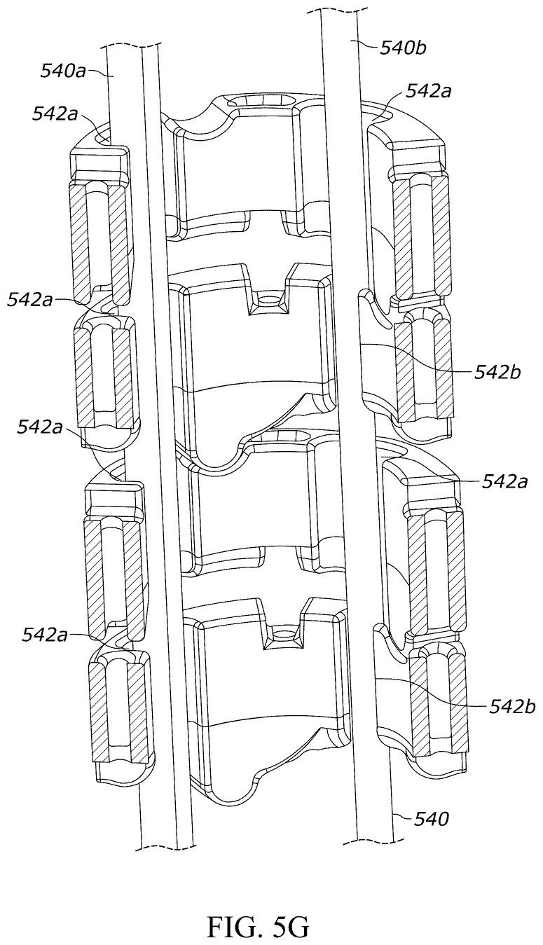

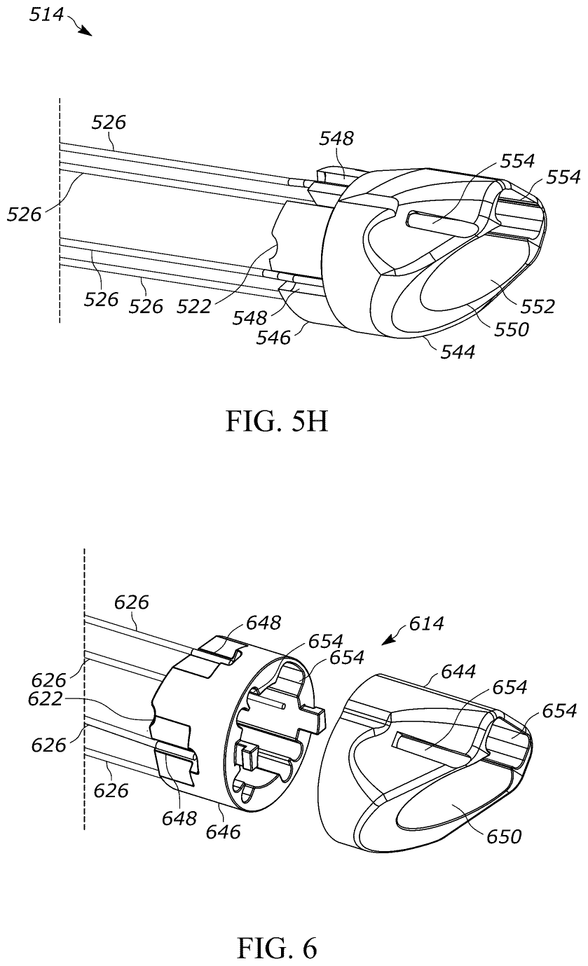

FIGS. 5A-5J illustrate components of a flexible elongate device 500 according to some embodiments. According to some embodiments consistent with FIGS. 1-4C, the flexible elongate device 500 may correspond to the elongate device 202 of the medical instrument system 200 and/or the flexible elongate device 400.

The flexible elongate device 500 includes a flexible body 502 extending from a proximal section 504 to a distal section 506. It will be understood that the flexible body 502, including the proximal and distal sections 504, 506, can have any desired length. Although not shown, the flexible body 502 can include an outer jacket that extends along some or all of a length of the flexible elongate device 500 to provide an outer surface for the flexible elongate device similar to the above example.

The flexible elongate device 500 includes an articulable body portion 510, which in some examples can be located in or form the distal section 506 of the flexible body 502, and an axial support structure 512 within the articulable body portion 510. The axial support structure 512 is configured to bend in response to articulation of the articulable body portion 510 and support the articulable body portion 510 against axial loads generated by articulation. In particular, the axial support structure 512 may prevent or reduce distortion, compression, and/or collapse of the articulable body portion 510 under axial loads. The flexible elongate device 500 can further include a distal member 514 disposed at a distal end of the axial support structure 512 and a proximal member 516 disposed at a proximal end of the axial support structure 512.

The axial support structure 512 includes a plurality of longitudinally stacked links 518. The axial support structure 512 can include any desired number of links 518 to provide an articulable body portion 510 of sufficient length. As shown in FIGS. 5B and 5C, each link 518 includes a body having a first end 518a and a second end 518b. To allow the axial support structure 512 to bend, each link 518 includes a hinge 520 protruding outwardly from the first end 518a of the body and a socket 522 at the second end 518b of the body configured to receive the hinge 520 of one of the links 518. The socket 522 is circumferentially offset relative to the hinge 520 by an angle, such that the links 518 can be stacked on one another with adjacent links 518 rotated relative to one another the amount of the circumferential offset. The axial support structure 512 can be bent by pivoting adjacent links 518 about a bending axis B (FIG. 5D) defined between the hinge 520 and socket 522 connection. This configuration allows adjacent pairs of links 518 of the axial support structure 512 to bend in different directions (e.g., along the bending axis B lateral to a longitudinal neutral axis). It will be understood that although the links 518 are shown with the hinges 520 oriented distally within the axial support structure 512, the links 518 can also have the reverse orientation with the hinges 520 oriented proximally.

An articulation angle a for adjacent links 518 can be a combined angle of adjacent end surfaces 524 of the adjacent links 518 (e.g., end surfaces 524 extending away from the hinge 520 and end surfaces 524 extending away from the socket 522) relative to a horizontal plane extending orthogonal to a longitudinal axis of the link 518. In other words, the end surfaces 524, as they extend laterally away from the hinge 520 and/or socket 522, can reduce an axial length of the link 518. This reduced axial length provides clearance for adjacent links 518 to pivot relative to one another about the hinge/socket interface. In one example as shown in FIGS. 5B and 5C, the end surface 524 of the first end 518a of the body is angled laterally away and downward from the hinge 520 relative to the horizontal plane and the end surface 524 of the second end 518b of the body is flat, extending generally along the horizontal plane. It will be understood that “angled laterally away” can include a continuous end surface as shown or can include discrete portions of the end surface configured to receive the adjacent end surface thereagainst when the links 518 are fully pivoted relative to one another. In other examples, both of the end surfaces 524 can be angled (e.g., the same or different angles) or the end surface 524 of the first end 518a can be flat, while the end surface 524 of the second end 518b can be angled laterally away and downward from the socket 522 relative to the horizontal plane. Additionally, although the end surfaces 524 are shown continuously extending between the hinge 520 or socket 522 (other than optional pull wire relief features described below), other embodiments can include additional recesses or cavities such that end surfaces 524 of adjacent links 518 only contact one another at discrete locations.

In some examples, articulation of the articulable body portion 510 can be controlled by a plurality of pull wires 526 that extend longitudinally along the flexible body 502. As such, each link 518 can receive the pull wires 526 therethrough. For example, each link 518 can define a plurality of pull wire openings 528, one pull wire opening 528 for each pull wire 526, that extend longitudinally through the body corresponding to the number and circumferential position of the pull wires 526. The pull wire openings 528 can take any suitable form. For example, the pull wire openings 528 can have a circular cross-section as shown, or can be oval or rectangular. In some examples, the pull wire openings 528 can be sized to closely match the dimensions of the pull wire 526. In some examples, the pull wire openings 528 can be sized to have clearance around one or more sides of the pull wire 526.

The flexible elongate device 500 can include any number of pull wires 526 to provide a desired amount of articulation. In some embodiments, the flexible elongate device 500 may include 2, 3, 4, 5, 6, or more pull wires 526 to articulate the articulable body portion 510. Three or four pull wires 526 can provide articulation along pitch and yaw axes. More than four pull wires 526 provide articulation along pitch and yaw axes in addition to providing additional functions or compounded geometry (e.g., S curvature). The angle of the circumferential offset of the hinge 520 and the socket 522 of each link 518 can be configured to accommodate desired articulation bending and/or patterns.

As shown, the hinge 520 and the socket 522 can be circumferentially offset relative to one another by 90 degrees for a four pull wire 526 configuration, such that every other pair of adjacent links 518 of the axial support structure 512 can bend along the same axis and the axial support structure 512 can bend along two axes (e.g., pitch and yaw). In other examples, the hinge 520 and the socket 522 can be circumferentially offset relative to one another by 120 degrees for a three pull wire 526 configuration, 72 degrees for a five pull wire 526 configuration, 60 degrees for a six pull wire 526 configuration, and so forth. Other suitable configurations for additional pull wires 526 can also be utilized.

To avoid or minimize (e.g., plastic) deformation of the pull wires 526, each of the links 518 can include relief features to accommodate the pull wires 526 during articulation of the articulable body portion 510. The relief features can be different depending on a location of the end of the pull wire opening 528 relative to the bending axis B of the adjacent pair of links 518. In one example as shown in FIGS. 5B-5E for four pull wires 526, each link 518 can include one or more recesses 530 defined at the end surfaces 524 of the link 518 aligned with pull wire opening ends that are (e.g., aligned) transverse to the adjacent hinge 520 or socket 522 (e.g., transverse to the bending axis B). In the illustrated form having four pull wires 526, each link includes two recesses 530 on each end. As shown in FIG. 5E, the aligned recesses 530 of the adjacent pair of links 518 provide a cavity through which the pull wire 526 can extend when the adjacent pair of links 518 are bent along the bending axis B. If the links 518 did not include the recesses 530, the pull wire 526 would be subjected to a more extreme angle during a (e.g., full) bend. In another example, as shown in FIGS. 5B-5D and 5F, the pull wire openings 528 of each link 518 can have an elongated configuration at an end thereof that is parallel to the hinge 520 or socket 522 (e.g., parallel to the bending axis B). In the illustrated embodiment, the elongated configuration is at an opposite end of the pull wire opening 528 from the recess 530, such that the pull wire 526 has relief at both ends of the link 518. In one example, the elongated configuration is provided by an inner surface 532 of the pull wire opening 528 having at least one expanding inner dimension (e.g., expanding to an increasingly elongated slot opening, such as by length and/or width, along a straight line or arcuate as shown) as the opening 528 extends to ends that are aligned generally parallel to the bending axis B of the adjacent hinge and socket interface. It will be understood that the terms “transverse” and “parallel”, when used to describe positioning relative to the hinge and socket interface may include a range of angles relative to the bending axis B. For example, the expanding inner dimension relief feature can be located to the side of the respective hinge/socket 520, 522 and still be considered to be aligned parallel with the bending axis B relative to the recesses 530, which are spaced a greater distance from the respective hinge/socket 520, 522 than the expanding inner dimension relief feature. In alternative embodiments, the angular location of the hinge/socket 520, 522 relative to the pull wire openings 528 may be more or less. For example, the pull wire openings 528 may be aligned directly with the hinge/socket 520, 522 interface or may be up to 45 degrees from the hinge/socket 520, 522 interface.

In some examples, the links 518 are not interlocked. As such, the links 518 would be susceptible to disengaging when tensile forces are applied to the axial support structure 512. To hold the links 518 in engagement with one another, the flexible elongate device 500 can include a braid sheath, such as the braid sheath 418 described above, having ends coupled (e.g., welded), to the distal member 514 and the proximal member 516. In another example, an antagonistic pair of pull wires 526 can be tensioned around a single capstan to hold the links 518 together. With this configuration, the braid sheath 418 may not be needed. Devices having one or more additional pairs of antagonistic pull wires 526 can include additional capstans for each antagonistic pair.

As shown in FIG. 5A, the flexible elongate device 500 can include an inner body member 534 that extends through the axial support structure 512. In this example, the links 518 define a central aperture 536 sized to receive the inner body member 534 therethrough and the hinge 520 and socket 522 includes portions aligned across the central aperture 536. For example, the links 518 can have a ring shape. Other examples, such as those without a central aperture, can be suitable for other steerable tools, e.g., a heart mapping catheter, or non-medical steerable tools, e.g., a borescope or plumber's scope.

In some examples, the hinge 520 can include inwardly tapering surfaces (e.g., tapering towards the first end 518a of the body of the link 518) that are configured to receive the socket 522 of an adjacent link 518 thereon. As shown, outwardly radial ends of the hinge 520 provide a highest axial height for the hinge 520 with the surfaces thereof tapering downwardly relative to the radial ends as the surfaces extend radially inwardly. The inwardly tapering surfaces are configured to center the adjacent link 518 when the socket 522 of the adjacent link 518 is disposed on the hinge 520 due to the socket 522 sliding relative to the hinge 520. In forms including the central aperture 536, the portions aligned across the central aperture 536 can each have the inwardly tapering surfaces, such as with a frustoconical or spherical configuration, and the socket 522 can have a complementary conical/spherical shape.

In some examples, the inner body member 534 can define or include a lumen 538 extending through the flexible body 502. The lumen 538 can provide a delivery channel for a medical tool, such as an endoscope, biopsy needle, endobronchial ultrasound (EBUS) probe, an imaging probe, ablation tool, electroporation tool, chemical delivery tool, a smaller flexible elongate device, and/or the like, to be inserted through the flexible body 502.

As shown in FIGS. 5A and 5G, the flexible elongate device 500 may include one or more component lines 540 extending along the length thereof. The component lines 540 may include components of the flexible elongate device 500 (e.g., a shape sensor) or portions of components located more distally (e.g., wiring for a sensor or other electronic device, illumination fiber for an imaging device, fluid tubing, etc.). In examples having multiple component lines 540, the lines 540 can run separately along the flexible elongate device 500 or be bundled together. The component lines 540 can have any desired functionality. In some examples, the flexible elongate device 500 may include one or more of: a shape sensor (e.g., fiber optic shape sensor) or other position/pose sensor(s), an imaging device, a wire, illumination fiber, etc. In the illustrated example, the flexible elongate device 500 includes a camera 540a and a shape sensor 540b.

The links 518 may advantageously define one or more component openings 542 that extend longitudinally through the body to allow the component lines 540 to extend through the articulable body portion 410. In some examples, it can be helpful for at least a plurality of the links 518 are identical for manufacturing purposes, however, due to the rotation necessitated by the hinge and socket offset, dedicated component openings would not align all of the way through the axial support structure 512. Accordingly, the links 518 can include a plurality of component openings 542 that are spaced from one another, such that when an adjacent link is rotated by the offset of the hinge and socket interface, one of the component openings 542 is always aligned along a component line path. In a further example, each link can include three component openings 542 spaced from one another such that two component openings for adjacent links 518 are always aligned to provide a longitudinal component line path through the axial support structure 512.

In some examples, the component openings 542 can have different configurations for different component lines 540. As shown in FIGS. 5B and 5C, each link 518 can include two elongated component openings 542a and one restricted size component opening 542b. With this configuration, one of the component line paths is defined by elongated openings 542a through the axial support structure 512 for components that require or can operate within an elongated space and the other component line path is defined in every other link 518 by the restricted size component opening 542b. For example, the shape sensor 520b (or other position sensor) can be routed through the component line path of alternating links having the restricted size component opening 542b to keep the position sensor 520a positionally secured through and with respect to the axial support structure 512. Advantageously, constraining the shape sensor 520 in every other link 518 sufficiently ensures accurate shape/position sensing, while also allowing all or a plurality of links 518 in the axial support structure 512 to have the same structure. Components, such as wiring for the camera 540a or illumination, that are not positionally sensitive relative to the axial support structure 512 can be routed through the pathway defined by the elongated component openings.

The component openings 542 can have any suitable cross-sectional shape, including, circular, oval, track-shaped, rectangular, square, and so forth. The component opening 542, as well as other edges of the links 518, can be chamfered. The component openings 542 can open into the central aperture 536 as shown, open through an exterior surface of the link 518, or not have an opening through the interior/exterior surfaces. In some examples, the component openings 542 can be configured to allow the component line(s) 540 to spiral around the circumference of the axial support structure 512.

The distal member 514 of the flexible elongate device 500 is shown in FIG. 5H. The distal member 514 includes a tip 544 and a control structure 546 (e.g., a control ring). In this example, the tip 544 and the control structure 546 are a single piece component (e.g., formed or irreleasably secured together by welding or the like). To control articulation of the articulable body portion 510 using the pull wires 526, at least two of the pull wires 526 are attached (e.g., welded, adhered, etc.) to the control structure 546 of the distal member 514 and, in some embodiments, all of the pull wires 526 are attached to the control structure 546. The pull wires 526 can be coupled to an exterior surface of the control structure 546 and/or the control structure 546 can define channels or recesses 548 to receive distal ends of the pull wires 526 therein. Further, the control structure 546 can include a socket 522 configured to receive the hinge 520 of a distal link 518 of the axial support structure 512 therein.

As shown, the distal member 514 can define a central aperture 550 that aligns with the central apertures 536 of the axial support structure 512. The central aperture 550 can receive the inner body member 534 or an extension sheath 552 therefor therein to provide a continuous passage through the distal end of the flexible elongate device 500. The distal member 514 can also define openings 554 for the components 540 aligned with the pathways through the axial support structure 512 to expose distal ends of the components 540 at a distal end of the flexible elongate device 500.

FIG. 6 shows a distal member 614 suitable for the flexible elongate device 500 in place of the distal member 514 thereof. In this form, the distal member 614 includes a tip 644 and a separate control structure 646 (e.g., a control ring) that is configured to couple to the tip 644. For example, the control structure 646 can snap fit to the tip 644, threadingly couple, and so forth. To control articulation of an articulable body portion (e.g., articulable body portion 510), in this form all pull wires 626 are attached (e.g., welded, adhered, etc.) to the control structure 646 of the distal member 614. The pull wires 626 can be coupled to an exterior surface of the control structure 646 and/or the control structure 646 can define channels or recesses 648 to receive distal ends of the pull wires 626 therein. Further, the control structure 646 can include a socket 622 configured to receive a hinge of a distal link of axial support structure (e.g., hinge 520, link 518, axial support structure 512) therein.

As shown, the distal member 614 can define a central aperture 650 that aligns with an aperture or lumen of the flexible elongate device (e.g., central apertures 536 of the axial support structure 512). The central aperture 650 can receive an inner body member (e.g., inner body member 534) or an extension sheath therein to provide a continuous passage through the distal end of the flexible elongate device. The distal member 614 can also define openings 654 for components of the flexible elongate device (e.g., components 540) aligned with component pathways through the axial support structure to expose distal ends of the components at a distal end of the flexible elongate device.

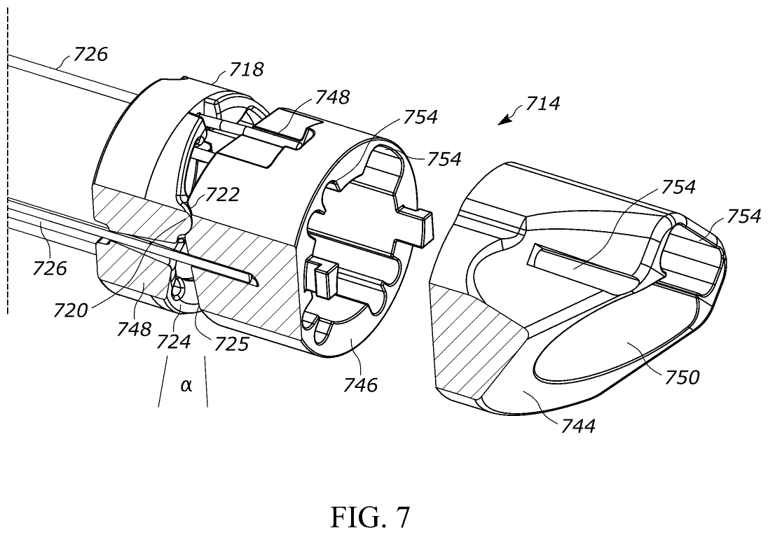

FIG. 7 shows a distal member 714 suitable for the flexible elongate device 500 in place of the distal member 514 thereof. The distal member 714 includes a tip 744 and a control structure 746 (e.g., a control ring). The tip 744 and control structure 746 can be separate components configured to couple together as shown as with the form of FIG. 6, or can be a single piece component as with the form of FIG. 5H.

In this example, some of pull wires 726 are attached to the control structure 746 and some pull wires 726 are attached to a distal link 718 suitable for the axial support structure 512 and having the structures described above with respect to the links 518. In the illustrated example of four pull wires 726, two opposing pull wires 726 are attached to the control structure 746 to control articulation about a first axis (e.g., pitch or yaw) and two opposing pull wires 726 are attached to the distal link 718 to control articulation about a second axis (e.g., pitch or yaw). The pull wires 726 can be coupled to an exterior surface of the control structure 746/distal link 718 and/or the control structure 746/distal link 718 can define channels or recesses 748 to receive distal ends of the pull wires 726 therein. Further, the control structure 746 can include a socket 722 configured to receive a hinge 720 of the distal link 718 therein.

As shown, the distal member 714 can define a central aperture 750 that aligns with an aperture or lumen of the flexible elongate device (e.g., central apertures 536 of the axial support structure 512). The central aperture 750 can receive an inner body member (e.g., inner body member 534) or an extension sheath therein to provide a continuous passage through the distal end of the flexible elongate device. The distal member 714 can also define openings 754 for components of the flexible elongate device (e.g., components 540) aligned with component pathways through the axial support structure to expose distal ends of the components at a distal end of the flexible elongate device.

An articulation angle a for adjacent links 718 and/or the distal link 718 and the control structure 746 can be a combined angle of adjacent end surfaces 724, 725 of the adjacent components (e.g., end surfaces 724 extending away from the hinge 720 and end surfaces 725 extending away from the socket 722) relative to a horizontal plane extending orthogonal to a longitudinal axis of the link 718/control structure 746. In other words, the end surfaces 724, 725, as they extend laterally away from the hinge 720 and/or socket 722, can reduce an axial length of the link 718/control structure 746. This reduced axial length provides clearance for adjacent links 718 to pivot relative to one another about the hinge/socket interface. In one example, shown in FIG. 7, the end surface 724 of the link 718 is angled laterally away and downward from the hinge 720 relative to the horizontal plane and the end surface 725 of the control structure 746 is angled laterally away and upward from the socket 722 relative to the horizontal plane. Although the control structure 746 is shown with the end surface 725 angled laterally away from the socket 722, any or all of the links 718 in the axial support structure can include similarly configured end surfaces 724. It will be understood that “angled laterally away” can include a continuous end surface as shown or can include discrete portions of the end surface configured to receive the adjacent end surface thereagainst when the links 718 are fully pivoted relative to one another. Additionally, although the end surfaces 724 are shown continuously extending between the hinge 720 or socket 722 (other than optional pull wire relief features described herein), other embodiments can include additional recesses or cavities such that end surfaces 724 of adjacent links 718 only contact one another at discrete locations.

In any of the above examples, one or more components of the flexible elongate device 400, 500 can include a friction reducing coating, layer, or component. For example, the links 518 can have any of the following materials disposed thereon: a parylene coating, a lubricant (e.g., oil) coating, a powder (e.g., molybdenum disulfide) coating, a polytetrafluoroethylene (PTFE) coating, chrome plating, a diamond-like-carbon (DLC) coating, a titanium nitride (TiN) coating, and/or a zirconium nitride coating. These materials can reduce friction between the hinges 522 and sockets 522 of the axial support structure 512 and between the links 518 and a braid sheath (e.g., braid sheath 418) disposed around the links 518. In some examples, the pull wire openings 528 can include a low friction sleeve or coating (e.g., any of the above materials) to reduce friction on the pull wires 526 as the pull wires 526 move with respect to the links 518 during articulation.

As set forth above, the combined angle of adjacent end surfaces 524 of adjacent links 518 is an articulation angle a for the links 518. In some examples, the axial support structure 512 can have the same articulation angle for all the links 518. In other examples, the axial support structure 512 can include a plurality of articulation angles, such as two, three, four, or more. Pursuant to this, a largest articulation angle for the axial support structure 512 can be provided by distal ones of the links 518 and/or the axial support structure 512 can include multiple, distally-increasing articulation angles.

In some examples, the links 518 can be made of a metal, such as stainless steel, titanium, or tungsten. In other examples, the links 518 can be made of a polymer. In other examples, the links 518 can be made from one or more materials that do not include nitinol.

In some examples, all of the links 518 in the axial support structure 512 are identical. In other examples, a distal link 518 of the axial support structure 512 has a shortened longitudinal length relative to the other links 518 in the axial support structure 512. In these examples, the links 518 other than the distal link 518 can be identical. In other or additional examples, the hinge/socket 520, 522 interface and end surfaces 524 can be configured to provide different overall bend angles in different bending planes.

In some examples, one or more of the links 518 can have a ground exterior surface. For example, the links 518 can be formed by metal injection molding and run through a grinding process. A stack of links 518 can be placed on a mandrel and ground down to remove wall thickness until the links 518 have a desired outer diameter. The grinding process can include centerless grinding. In these examples, a surface of the central aperture 536 of the links 518, if included, can be left in a rough, unground condition.

In any of the examples provided herein, a diameter of the central aperture 536 of the links can be between about 2.45 mm to about 2.8 mm; an outer diameter of the links 518 can be between about 3.81 mm to about 3.66 mm; a thinnest wall section of the links 518 can be between about 0.15 mm to about 0.23 mm; and/or a thickest wall section of the links 518 can be between about 0.43 mm to about 0.61 mm.

In any of the examples provided herein, the axial support structure 512 can include up to 25 links 518 or more to provide a bending plane articulation of at least 185 degrees and a 45 degrees off bending plane articulation of at least 262 degrees; and/or the axial support structure 512 may allow a 12 mm bend radius or more (e.g., a bending plane radius of at least 13.7 mm and a 45 degrees off bending plane radius of at least 9.7 mm).

In any of the examples provided herein, the hinge 522 to socket 520 height can be between about 0.35 mm to about 0.38 mm; and/or the hinge 520 inwardly tapering surfaces can taper at an angle of between about 11.5 degrees to about 12.5 degrees.

One or more components of the embodiments discussed in this disclosure, such as control system 112, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

1. A flexible elongate device comprising:
an elongate body having an articulable body portion; and
an axial support structure within the articulable body portion, the axial support structure comprising a plurality of links longitudinally stacked on one another, each of the plurality of links comprising:
a body having a first end and a second end, the body defining a plurality of pull wire openings extending therethrough to receive a plurality of pull wires that control articulation of the articulable body portion;
a hinge protruding outwardly from the first end of the body;
a socket at the second end of the body, the socket being circumferentially offset relative to the hinge and configured to receive the hinge of one of the links therein;
a first pull wire relief recess at the first end of the body aligned with a first pull wire opening that is transverse to the hinge; and
a second pull wire relief recess at the second end of the body aligned with a second pull wire opening that is transverse to the socket.

2. The flexible elongate device of claim 1, wherein the first pull wire opening has an elongated configuration at the second end of the body that is parallel to the socket; and the second pull wire opening has an elongated configuration at the first end of the body that this parallel to the hinge.

3. The flexible elongate device of claim 1, wherein the hinge includes inwardly tapering surfaces configured to center an adjacent link of the plurality of links when the socket of the adjacent link is disposed on the hinge.

4. The flexible elongate device of claim 1, wherein the first end of the body has an end surface angled away from the hinge, the second end of the body has an end surface angled away from the socket, or both.

5. The flexible elongate device of claim 1, wherein the body of each of the plurality of links define three component openings extending therethrough, adjacent links of the plurality of links having two component openings that align to provide a longitudinal path through the axial support structure.

6. The flexible elongate device of claim 5, wherein the three component openings include two elongated component slots and a cylindrical opening, the cylindrical openings of the plurality of links providing a restricted longitudinal path through the axial support structure; and further comprising:
a first component line comprising a shape sensor received with the restricted longitudinal path through the axial support structure; and
a component line received the other longitudinal path.

7. (canceled)

8. The flexible elongate device of claim 1, wherein the hinge and socket are offset relative to one another by an angle corresponding to the plurality of pull wires to allow articulation of the articulable body portion along axes for each of the plurality of pull wires.

9. The flexible elongate device of claim 8, wherein the hinge and socket are offset 90 degrees relative to one another, such that the plurality of pull wires control articulation of the articulable body portion along pitch and yaw axes; or are offset 120 degrees relative to one another.

10. (canceled)

11. The flexible elongate device of claim 1, wherein each link of the plurality of links defines an aperture; and the hinge includes opposing frustoconical or spherical portions aligned across the aperture.

12-13. (canceled)

14. The flexible elongate device of claim 1, wherein at least one of the plurality of pull wire openings or the plurality of links include a low friction sleeve or coating.

15. (canceled)

16. The flexible elongate device of claim 1, wherein an articulation angle for adjacent links of the plurality of links is a combined angle of adjacent end surfaces of the adjacent links.

17. The flexible elongate device of claim 16, wherein one of the end surfaces of the plurality of links is flat.

18. The flexible elongate device of claim 16, wherein the axial support structure includes a plurality of articulation angles, and a largest articulation angle of the axial support structure is provided by distal links of the plurality of links.

19-22. (canceled)

23. The flexible elongate device of claim 1, wherein the plurality of links are not interlocked.

24. The flexible elongate device of claim 1, wherein the articulable body portion further comprises a distal member and a proximal member, the plurality of links stacked longitudinally stacked on one another between the distal member and the proximal member; and further comprising a braid sheath having ends coupled to the distal member and the proximal member to hold the plurality of links in engagement with one another.

25. (canceled)

26. The flexible elongate device of claim 1, wherein each of the plurality of links are identical.

27. (canceled)

28. The flexible elongate device of claim 1, wherein the articulable body portion includes a control structure disposed distally of the axial support structure, at least two of the plurality of pull wires coupled to the control structure.

29. The flexible elongate device of claim 28, wherein at least two of the plurality of pull wires are coupled to a distal link of the plurality of links.

30. The flexible elongate device of claim 29, wherein the distal link has a shortened longitudinal length relative to other links of the plurality of links.

31. (canceled)

32. The flexible elongate device of claim 1, wherein each of the plurality of links has a ground exterior surface.

33-60. (canceled)
